(19) United States
(12) Patent Application Publication
(10) Pub. No.: US 2022/0151892 A1
(43) Pub. Date: May 19, 2022

(54) A HAIR STRAIGHTENING OR HAIR STYLING COMPOSITION AND METHOD OF USE THEREOF

(71) Applicant: ISP INVESTMENTS LLC, Wilmington, DE (US)

(72) Inventors: Manuel GAMEZ-GARCIA, New City, NY (US); Yan ZHOU, Branchburg, NJ (US)

(73) Assignee: ISP INVESTMENTS LLC, Wilmington, DE (US)

(21) Appl. No.: 17/439,662

(22) PCT Filed: Mar. 20, 2021
(86) PCT No.: PCT/US2020/022900

§ 371 Date: Sep. 15, 2021

Related U.S. Application Data
(60) Provisional application No. 62/819,179, filed on Mar. 15, 2019.

(51) Int. Cl. A61K 8/362; A61K 8/34; A61K 8/35; A61Q 5/04; A61Q 5/06

(52) U.S. Cl. CPC A61K 8/362; A61K 8/34; A61Q 5/06; A61Q 5/04; A61K 8/35

(57) ABSTRACT

A hair straightening or styling composition provided herein comprises: (i) citraconic acid; (ii) (a) a reducing agent and/or (b) an aldehyde, a thioacetal, a ketone or an alcohol; (iii) a buffering agent; (iv) an oxidizing agent; (v) optionally a carboxylic or dicarboxylic acid; and (vi) at least one solvent. Also provided herein is a method of straightening or styling hair using such composition.

### FIELD OF THE INVENTION

The present application relates to a hair straightening or styling composition and to a method of using the composition in styling or straightening human hair.

### BACKGROUND OF THE INVENTION

It is well known that the process for changing the shape of hair from curly to straight poses several difficult challenges. Among those, three important challenges include (i) safety of the active ingredient employed; (ii) good straightening efficiency of hair; and (iii) the need for a practical formulation having a robustness during the hot ironing process. Over the past years, various techniques to straighten curly hair have been proposed, however, none address satisfactorily all these challenges.

Canadian Patent Publication CA1081125A [Assigned to Helene Curtis Industries Inc.] and U.S. Pat. No. 7,622,104 [Assigned to L'Oreal SA] disclose a hair straightening method called reverse perming wherein, thioglycolic acid derivatives or sulfites are used to break disulfide bonds followed by a process of bond reformation via an oxidizing agent, usually hydrogen peroxide. Unwanted thiol odors are produced and are involved multiple stages.

U.S. Pat. No. 4,314,572A [Assigned to Carson Products Co.] discloses a hair straightening process wherein disulfide bonds in hair are broken using alkali creams based on sodium hydroxide, lithium hydroxide, or guanidinium hydroxide at high pH followed by addition of citric acid to neutralize the alkalinity in hair.

US Patent Publication US2006/0104928A1 [Assigned to Lucimar Furtado] discloses use of formaldehyde in hair straightening methods. Formaldehyde, however, is known to be highly toxic and therefore this active has gradually been replaced by glyoxylic acid or glyoxylic acid derivatives which are good straightening agents.

PCT Publication WO2018/122123A1 [Assigned to L'Oreal] discloses a process for treating keratin fibers by applying a composition comprising butene dioic acid and/or its derivatives and amino silicones to hair fibers followed by hair treatment at a temperature equal to 100° C.

In view of the foregoing, there still exists a need in the art for an efficient method that (i) successfully straightens hair without causing any irritation to the user's scalp, (ii) lasts long and sustains styled or straightened hair tresses after multiple shampoo wash cycles.

### SUMMARY OF THE INVENTION

The applicants have now discovered that citraconic acid with reducing agents, aldehydes, ketones, alcohols, alone or in combination results in effective hair straightening, thereby making it possible to overcome the disadvantages of the prior art.

One aspect of the present application provides a hair straightening or hair styling composition comprising: (i) from 0.5 to 60 wt. % of citraconic acid; (ii)(a) from 0.01 to 5 wt. % of a reducing agent; and/or (b) from 0.001 to 50 wt. % of (b1) an aldehyde, (b2) a thioacetal, (b3) a ketone, (b4) an alcohol, or combinations thereof; (iii) from 0.1 to 20 wt. % of a buffering agent; (iv) from 0.01 to 8 wt. % of an oxidizing agent; (v) from 0.0 to 5 wt. % of a carboxylic or a dicarboxylic acid; and (vi) from 2 to 20 wt. % of at least one solvent.

In another aspect, the present application provides a hair straightening or hair styling composition comprising: (i) from 0.5 to 60 wt. % of citraconic acid; (ii)(a) from 0.01 to 5 wt. % of a reducing agent selected from thioglycolic acid, thiolactic acid, their salts, or bisulfites; and/or (b) from 0.001 to 50 wt. % of (b1) an aldehyde selected from butyraldehyde, iso-butyraldehyde, or propionaldehyde, (b2) a thioacetal. (b3) cyclopentanone, (b4) an alcohol selected from isopropyl alcohol or propylene glycol or combinations thereof; (iii) from 0.1 to 20 wt. % of a buffering agent selected from sodium acetate, sodium diacetate or disodium succinate; (iv) from 0.01 to 5 wt. % of an oxidizing agent selected from hydrogen peroxide, urea/peroxide, calcium peroxide or magnesium peroxide; (v) from 0.0 to 5 wt. % of a carboxylic or dicarboxylic acid; and (vi) from 2 to 20 wt. % of at least one solvent selected from ethylene carbonate and water.

In yet another aspect, the present application provides a method of straightening or styling hair, the method comprising: (a) washing hair tresses with shampoo; (b) drying the hair with a blow dryer in a temperature range from 40-80′C; (c) applying the above described hair styling or hair straightening composition to hair: (d) leaving the hair composition onto the hair for a period of 20 to 40 min; (e) removing the excess formulation from the hair; (f) applying from 5 to 15 hot ironing strokes to the hair, wherein the hot iron is set above the hair glass transition temperature (145° C.) and denaturation temperature (156° C.), between 160 to 220° C.; (g) optionally, washing the hair with a shampoo; and (h) obtaining the straightened or styled hair.

### BRIEF DESCRIPTION OF THE DRAWINGS

Objects, features, and advantages of the present invention will become apparent upon reading the following description in conjunction with the drawings/figures, in which:

FIG. 1 is an illustration of various hair tresses treated with actives, straightened at 450° F. with hot iron and then subjected to 20 shampoo wash cycles.

FIG. 2 is an illustration of various hair tresses treated with cream formula—A disclosed in Example 27. Photographs refer to: (A) Untreated not hot ironed control, (B) Hot ironed and treated with formula A, (C) Hair tresses after 5 shampoo washings, (D) After 15 shampoo washings, and (E) After 20 shampoo washings respectively.

FIG. 3 is an illustration of various hair tresses treated with mousse base formula—B disclosed in Example 28. Photographs refer to: (A) Untreated not hot ironed control, (B) Hot ironed and treated with formula A, (C) Hair tresses after 5 shampoo washings, (D) After 15 shampoo washings, and (E) After 20 shampoo washings respectively.

### DETAILED DESCRIPTION OF THE INVENTION

Before explaining embodiment(s) of the present disclosure in detail, it is to be understood that the present disclosure is not limited in its application to details of construction and the arrangement of the components or steps or methodologies set forth in the following description or illustrated in the figures. The present disclosure is capable of other embodiments or of being practiced or carried out in various ways. Also, it us to be understood that the phraseology and terminology employed herein is for the purpose of description and should not be regarded as limiting. The following detailed discussion of the various alternative and preferred embodiments will illustrate the general principles of the invention. The following detailed description is intended to be representative only and not limiting as to the described inventive elements. Many variations can be derived by one skilled in the art which are included within the scope of the present invention.

Unless otherwise defined herein, technical terms used in connection with the present disclosure shall have the meanings that are commonly understood by those of ordinary skill in the art. Further, unless otherwise required by context, singular terms shall include pluralities and plural terms shall include the singular.

All percentages, parts, proportions and ratios as used herein, are by weight of the total composition, unless otherwise specified. All such weights as they pertain to listed ingredients are based on the active level and, therefore; do not include solvents or byproducts that can be included in commercially available materials, unless otherwise specified.

All publications, articles, papers, patents, patent publications, and other references cited herein are hereby incorporated herein in their entirety for all purposes to the extent consistent with the disclosure herein.

All references to singular characteristics or limitations of the present disclosure shall include the corresponding plural characteristic or limitation, and vice-versa, unless otherwise specified or clearly implied to the contrary by the context in which the reference is made.

As utilized in accordance with the present disclosure, the following terms, unless otherwise indicated, shall be understood to have the following meaning.

The use of the word “a” or “an” when used in conjunction with the term “comprising” may mean “one,” but it is also consistent with the meaning of “one or more, at least one,” and “one or more than one.” The use of the term “or” is used to mean “and/or” unless explicitly indicated to refer to alternatives only if the alternatives are mutually exclusive, although the disclosure supports a definition that refers to only alternatives and “and/or.” The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results. In addition, the use of the term “at least one of X, Y. and Z” will be understood to include X alone, Y alone, and Z alone, as well as any combination of X, Y, and Z. The use of ordinal number terminology (i.e., “first”, “second”, “third”, “fourth”, etc.) is solely for the purpose of differentiating between two or more items and, unless otherwise stated, is not meant to imply any sequence or order or importance to one item over another or any order of addition.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “or combinations thereof” as used herein refers to all permutations and combinations of the listed items preceding the term. For example, “A, B, C, or combinations thereof” is intended to include at least one of: A, B, C, AB, AC, BC, or ABC and, if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Numerical ranges as used herein are intended to include every number and subset of numbers contained within that range, whether specifically disclosed or not. Further, these numerical ranges should be construed as providing support for a claim directed to any number or subset of numbers in that range.

The term “active” as used herein, refers to any ingredient used in the preparation of the hair straightening or hair styling composition of this invention. Actives include (i) citraconic acid; (ii) reducing agent such as, aldehyde, thioacetal, ketone, alcohol; (iii) buffering agent; (iv) oxidizing agent; and (v) carboxylic or dicarboxylic acid.

The term “cosmetically acceptable ingredient” means any ingredient/compound or mixture of ingredients/compounds or compositions that are typically employed to produce other aesthetic or cosmetic based desirable effects in personal care compositions. Cosmetically, acceptable excipients include, but are not limited to, preservatives, antioxidants, chelating agents, sunscreen agents, proteins, amino acids, vitamins, dyes, hair coloring agents, plant extracts, humectants, fragrances, emollients, lubricants, butters, penetrants, thickeners, viscosity modifiers, polymers, resins, hair fixatives, film formers, surfactants, detergents, emulsifiers, pacifying agents, volatiles, propellants, liquid vehicles, carriers, salts, pH adjusting agents, neutralizing agents, hair conditioning agents, anti-static agents, anti-frizz agents, anti-dandruff agents, hair waving agents, relaxers, absorbents, fatty substances, gelling agents, moisturizers, hydrophilic or lipophilic active agent, fillers, reducing agents, cosmetic oils, perfumes, liquid vehicles, solvents, and combinations thereof.

The term “hair straightening” and “hairstyling” as used herein, are interchangeable. Both terms refer to the straightening or changing the shape of hair by using the active agents.

The term “hair straightening” refers to the state where the natural curl of the hair has been removed and resulting straight configuration effectively resists reversion (i.e. resumption of a curly pattern) on being subjected to more than at least one washing, preferably, at least four washings, and more preferably at least eight washings or until the scalp hair grows sufficiently to visibly warrant another hair straightening procedure.

The term “hair straightening iron” refers to a device used in association with a combination of heat and pressure to straighten hair. It is generally composed of two flat heating elements, covered with various materials, between which a lock of hair is pressed. Hair straightening irons are commercially available and are known to a person skilled in the relevant art. The source of energy of heating can be electric, solar, light, magnetic, chemical, electromagnetic or combinations thereof.

The term “thermally activated agent” as used herein, refers to any agent activated by heat using a heating device. The device can be a hot iron or other device heated to a temperature of at least 175° C. for a sufficient time to modify the keratin fibers. By “thermally-activated” it is meant that the agents are activated by heat, so they can react with the internal protein structure of hair to induce the desired changes in its shape.

Applicants have now discovered that citraconic acid alone or in combination with reducing agents, aldehydes, thiol group containing compounds, thioacetals, and/or ketones and/or alcohols is effective in straightening or styling hair.

In one embodiment, the present application provides a hair straightening or hair styling composition comprising: (i) from 0.5 to 60 wt. % of citraconic acid; (ii) (a) from 0.01 to 5 wt. % of a reducing agent and/or (b) from 0.001 to 50 wt. % of (b1) an aldehyde, (b2) a thioacetal, (b3) a ketone, (b4) an alcohol, or combinations thereof; (iii) from 0.1 to 20 wt. % of a buffering agent; (iv) from 0.01 to 5 wt. % of an oxidizing agent; (v) from 0.0 to 5 wt. % of a carboxylic or dicarboxylic acid; and (vi) from 2 to 20 wt. % of at least one solvent.

It has been surprisingly discovered that citraconic acid acts as a water co-solubilizing agent for hydrophobic aldehydes and other hydrophobic agents. The range of citraconic acid present in the synergistic composition is from 0.5 wt. % to 60 wt. %, preferably from 5 wt. % to 40 wt. % and more preferably between 10 to 30 wt. % of the total composition.

In one embodiment, the citraconic acid [IUPAC Name: (2Z)-2-Methylbut-2-enedioic acid] is a white solid organic compound having a formula CH₃C₂H(CO₂H)₂. It is the cis-isomer of mesaconic acid having following structure:

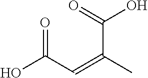

The molecular weight of citraconic acid is 130.10, it has a density of 1.62, and a melting point of 90° C. The evaluated samples of this compound had a purity of 98%.

According to another embodiment, the reducing agent is selected from the group consisting of thioglycolic acid, thiolactic acid and their salts, sodium bisulfite, sodium sulfite, cysteine, or thioacetals formed by the reaction of aldehydes and thio compounds, either, in situ or prior to formulation.

According to another embodiment, the aldehyde is selected from the group consisting of acetaldehyde, propionaldehyde, butyraldehyde, iso-butyraldehyde, glyoxal, glutaraldehyde, pyruvaldehyde, methylglyoxal di-acetate, glyoxylic acid, 5-methyl furaldehyde, cinnamaldehyde, benzaldehyde, hydroxyaldehyde, cyclohexanecarbaldehyde, (R)-2-chloro-3-phenylpropanal, 2-methyl-3-oxopentanal, 4-methyl-3-oxopentanal, 2-(3-ethylbenzoyl)-3-oxopentanal, 2,2,4-trimethyl-3-oxopentanal, 2,2-dimethyl-3-oxopentanal, 4-oxopentanal, anisaldehyde, salicylaldehyde, hydroxybenzaldehyde, hemiacetals, and thioacetals formed in situ or previous to formulation by the reaction of other aldehydes or by compounds containing a thiol group present in the mixture.

According to another embodiment, the ketone is selected from the group consisting of acetone, mono-hydroxyacetone, dihydroxy acetone, cyclopentanone, hexanone, caprolactone, valerolactone, methyl furyl ketone, methyl isobutyl ketone, and raspberry ketone.

According to another embodiment, more preferably, the alcohol is selected from the group consisting of phenoxyethanol, ethanol, methanol, propanediol, propylene glycol, octane diol, and butylene glycol.

The range of the aldehyde, ketone, or alcohol is from 0.1 wt. % to 50 wt. %, preferably from 1.0 wt. % to 30 wt. % and more preferable from 0.05 wt. % to 10 wt. %.

According to another embodiment, the buffering agent is selected from the group consisting of sodium diacetate, sodium acetate, sodium succinate, disodium succinate, sodium citrate, sodium formate, and sodium or ammonium salts formed in situ by the neutralization of acetic acid, succinic acid, citric acid, sodium carbonate, or formic acid.

According to another embodiment, the oxidizing agent is selected from the group consisting of hydrogen peroxide, benzoyl peroxide, magnesium peroxide, calcium peroxide, dicumyl peroxide, urea peroxide, sodium persulfate, ammonium persulfate, sodium percarbonate, sodium bromates, cyclopentanone, magnesium oxide, and zinc oxide.

According to another embodiment, the solvent is selected from group consisting of isopropyl alcohol, propylene glycol, propanediol, glycerin, methyl acetate, ethanol, acetone, amyl acetate, ethyl dimethyl dioxolane, ethylene carbonate, dimethyl carbonate, phenoxyethanol, acetyl-methylfurfural, butylene glycol, PEG200, PEG400, diethylene glycol and trimethyl phosphate.

According to another embodiment, the optional carboxylic acids are selected from, but not limited to, the group consisting of mesaconic acid, itaconic acid, phosphoric acid, acetic acid, citric acid, malonic acid, succinic acid and formic acid.

The total amount of actives in the formulation is from 0.5 wt. % to 50 wt. %, preferably from 5 wt. % to 40 w. % more preferably, from 10 wt. % to 30 wt. %. The styling composition of the present application has a pH of 1.5 to 3.0; preferably, 2.0 to 2.7 and more preferably, 2.0 to 2.57. The composition of the present application shows at least 60% efficacity of straightening or styling lasting for 20 shampoo wash cycles. The hair straightening, or hair styling composition can be formulated into a cream, a mousse, a foam, a serum, a lotion paste, a hair gel, a hair pudding, or a spray.

The formulation comprises at least one cosmetically acceptable ingredient including, but not limited to, a cationic, an anionic, or non-ionic polymer, a conditioning agent selected from stearamidopropyl amine and fatty quaternaries, an oil, an emollient, a humectant, a petrolatum thickener, a mineral oil, an emulsifier, a fragrance, a keratin, a protein or a preservative. All these ingredients can be selected to provide the appropriate consistency and stability to the formulation.

The hair straightening or hair styling composition may further comprise an anionic surfactant, a non-ionic surfactant, amphoteric surfactant or a cationic surfactant.

In another embodiment, the present application provides a hair straightening or hair styling composition comprising: (i) from 0.5 wt. % to 60 wt. % of citraconic acid; (ii) (a) from 0.01 wt. % to 5 wt. % of a reducing agent selected from thioglycolic acid, thiolactic acid, thioacetals, and bisulfites and/or (b) from 0.001 wt. % to 50 wt. % of (b1) an aldehyde selected from butyraldehyde, iso-butyraldehyde, propionaldehyde, benzaldehyde, and anisaldehyde; (b2) a thioacetal, (b3) a ketone selected from cyclopentanone, (b4) an alcohol selected from isopropyl alcohol or propylene glycol, or combinations thereof; (iii) from 0.1 wt. % to 20 wt. % of a buffering agent selected from sodium diacetate, sodium acetate, and disodium succinate; (iv) from 0.01 wt. % to 5 wt. % of oxidizing agent selected from hydrogen peroxide, urea/peroxide, calcium peroxide and magnesium oxide; (v) optionally, up to 5 wt % of a carboxylic or dicarboxylic acid; and (vi) from 2 wt. % to 20 wt. % of at least one solvent selected from water and ethylene carbonate.

In yet another embodiment, the present application provides a method of straightening or styling hair, the method comprising: (a) washing hair tresses with shampoo; (b) drying the hair with a blow dryer in a temperature range from 40-80° C.; (c) applying to the hair the hair styling or hair straightening composition comprising: (i) from 0.5 wt. % to 60 wt. % of citraconic acid; (ii) (a) from 0.01 wt. % to 5 wt. % of a reducing agent and/or (b) from 0.001 wt. % to 50 wt. % of (b1) an aldehyde, (b2) a thioacetal. (b3) a ketone, or (b4) an alcohol; (iii) from 0.1 wt. % to 20 wt. % of buffering agent; (iv) from 0.01 wt. % to 5 wt. % of an oxidizing agent; (v) from 0.0 to 5 wt. % of a carboxylic or a dicarboxylic acid; (vi) from 2 wt. % to 20 wt. % of a solvent, (d) leaving the hair composition onto the hair for a period of 20 to 40 min; (e) removing the excess formulation from the hair; (f) applying from 5 to 15 hot ironing strokes to the hair, wherein the hot iron is set above the hair glass transition temperature (145° C.) and denaturation temperature (156° C.), between 160 to 220° C.; (g) optionally, washing the hair with a shampoo; and (h) obtaining the straightened or styled hair.

The hairstyling or hair straightening composition can be formulated in the form of a cream, foam, a serum, a lotion paste, a hair gel, a hair pudding, a spray, or a mousse. The composition can also be formulated in separate parts A and B in the form of creams, lotions, mousses, serums, hair gels, hair puddings, sprays, etc. Then, before application, Part A and B can be combined. The part containing the citraconic acid is referred to as the activator.

The hair tresses employed for straightening or styling can be Caucasian, Asian, or African in origin selected from fine, medium and coarse virgin, damaged, colored, grey or bleached hair. These tresses further can have visibly wavy, curly, tightly curly or kinky configuration.

The extent to which the hair treated with actives has been straightened or styled is measured in “The efficacy values” (abbreviated as “Eff”) calculated according to the following equation (1):

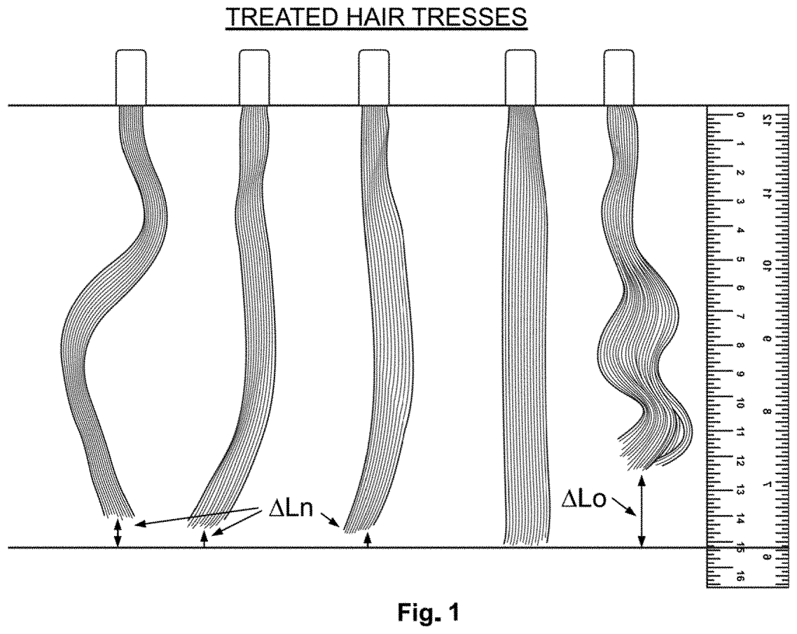

Eff=(ΔL1)×100/ΔLo Equation (1):

where ΔL1 is the difference in length of the hair tress measured in its fully straightened shape before shampooing minus the length of the hair tress measured after it has been hot ironed and shampooed 20 times (see FIG. 1). ΔLo is the difference in hair length of the hair tress measured immediately after hot ironing without shampooing minus the length of the hair tress in its initial curly state.

Without wishing to be bound by theory, it is believed that the synergistic straightening effects arise from the fact that when all actives described above are heated inside the hair cortex they follow a series of physical chemical processes which occur in a timed sequence and favor the permanent straightening of curly hair. Hair straightening or styling occurs in three steps. The first step is hair fiber softening involving thermal, physical, or chemical bond breakage. During any hot ironing process, fiber softening is achieved by heating the hair above its glass transition and denaturation temperatures, i.e. above ˜156° C. The second step is shape imposition and mechanical stress release. Shape imposition on hair is achieved by the compression and tension of the hot iron plates. The third step is bond reformation or new bond creation to stabilize the newly formed shape. In the case of our invention, the softening of hair is believed to be further assisted by the presence of small amounts of reducing agents and/or aldehydes which will enhance keratin plasticization by bond breakage reformation during heating. Finally, bond creation to lock-in the new straight shape is accomplished by protein crosslinking due to the reaction of heat activated citraconic acid. In fact, these three processes can occur within the hair cortex at slightly different times or almost simultaneously during hot ironing. However, no matter how close in time they can occur, it is believed that the final step of crosslinking occurs after keratin softening and after the straight shape has been imposed, otherwise, the change in shape will be inefficient. It is further believed that the synergy observed with citraconic acid in combination with a small amount of reducing agent and/or aldehydes, and/or ketones, and/or alcohols, and or oxidizing agents, results from their appropriate selection and concentration which then not only results in a favorable protein softening/crosslinking sequence but also in its timely occurrence.

The following examples presented herein are for purposes of illustration of the present disclosure and not intended to be limiting, for example, the combination of citraconic acid with other actives in hair straightening and hairstyling compositions can also be extended to various other personal care applications.

### EXAMPLES

Examples 1-6: The examples detailed below in Table I lists hair straightening efficiencies (Effs), showing that when citraconic acid is combined with a reducing agent at low percentage levels, a solvent (ethylene carbonate), and a buffer (sodium diacetate), the efficiency levels (Effs) of hair straightening improves substantially due to synergy of these ingredients.

TABLE I
Citraconic acid alone or in synergistic combination with other actives

| Ingredients & | EXP 1 | EXP 2 | EXP 4 | EXP 5 | EXP 6 |
|---|---|---|---|---|---|
| Evaluation | (wt. %) | (wt. %) | (wt. %) | (wt. %) | (wt. %) |
| Citraconic acid | 15% | 15% | 15% | 15% | 15% |
| Butyraldehyde | — | — | — | — | — |
| Iso-butyraldehyde | — | — | — | — | — |
| Propionaldehyde | — | — | — | — | — |
| Phenoxyethanol | — | — | — | — | — |
| Cyclopentanone | — | — | — | — | — |
| Ethylene carbonate | — | 4.0% | 4.0% | 4.0% | 4.0% |
| Sodium Diacetate | 10% | 10% | 10% | 10% | 10% |
| Isopropyl alcohol | — | — | — | — | — |
| Propylene glycol | — | — | — | — | — |
| Thioglycolic acid | — | 1.5% | — | — | — |
| Thiolactic acid | — | — | 1.5% | — | — |
| Hydrogen peroxide | — | — | — | — | — |
| Magnesium oxide | — | — | — | — | — |
| Water to 100 | BAL | BAL | BAL | BAL | BAL |
| pH (H₃PO₄) | 2.6 | 2.6 | 2.6 | 2.6 | 2.87 |
| % Straightening | 30% | 100% | 100% | 56% | 45% |

### Comparative Examples 7-12

TABLE II
Without Citraconic acid in combination with other actives

| | EXP | EXP | EXP | EXP | EXP | EXP |
|---|---|---|---|---|---|---|
| Ingredients & | 7 | 8 | 9 | 10 | 11 | 12 |
| Evaluation | (wt. %) | (wt. %) | (wt. %) | (wt. %) | (wt. %) | (wt. %) |
| Citraconic acid | — | — | — | — | — | — |
| Butyraldehyde | — | 5% | — | — | — | — |
| Iso-butyraldehyde | — | — | 5% | — | — | — |
| Propionaldehyde | — | — | — | 5% | — | — |
| Phenoxyethanol | — | — | — | — | 5% | — |
| Cyclopentanome | — | — | — | — | — | 5% |
| Sodium Diacetate | 10% | 10% | 10% | 10% | 10% | 10% |
| Isopropyl alcohol | — | — | — | — | — | — |
| Propylene glycol | — | — | — | — | — | — |
| Hydrogen | — | — | — | — | — | — |
| peroxide | | | | | | |
| Magnesium oxide | — | — | — | — | — | — |
| Water to 100 | BAL | BAL | BAL | BAL | BAL | BAL |
| pH | 2.6 | 2.6 | 2.6 | 2.6 | 2.6 | 2.87 |
| % Straightening | 30% | 45% | 60% | 63% | 56% | 45% |

Table II shows straightening efficiency values of various aldehydes, ketones, and alcohols obtained when citraconic acid is absent in the formulations and as it can be in this Table. Most straightening efficiency values decreased substantially to values as low as 50% (see Table II). Both observations clearly indicate that the combination of citraconic acid with the selected aldehydes, ketones, and alcohols shown in the examples leads to hair straightening synergistic effects.

### Examples 13-21

TABLE III
Citraconic acid alone or synergistic combination with other actives

| Ingredients & | EXP 13 | EXP 14 | EXP 15 | EXP 16 | EXP 17 | EXP 18 | EXP 19 | EXP 20 | EXP 21 |
|---|---|---|---|---|---|---|---|---|---|
| Evaluation | (wt. %) | (wt. %) | (wt. %) | (wt. %) | (wt. %) | (wt. %) | (wt. %) | (wt. %) | (wt. %) |
| Citraconic acid | 20% | 20% | 20% | 20% | 20% | 20% | 20% | 20% | 20% |
| Butyraldehyde | — | 5% | — | — | — | — | 2.5% | — | — |
| Iso-butyraldehyde | — | — | 5% | — | — | — | — | 2.5% | 2.5% |
| Propionaldehyde | — | — | — | 5% | — | — | 2.5% | — | — |
| Phenoxyethanol | — | — | — | — | 5% | — | — | — | 2.5 |
| Cyclopentanome | — | — | — | — | — | 5% | — | 2.5 | — |
| Sodium Diacetate | 10% | 10% | 10% | 10% | 10% | 10% | 10% | 10% | 10% |
| Isopropyl alcohol | — | — | — | — | — | — | — | — | — |
| Propylene glycol | — | — | — | — | — | — | — | — | — |
| Hydrogen | — | — | — | — | — | — | — | — | — |
| Peroxide | | | | | | | | | |
| Magnesium Oxide | — | — | — | — | — | — | — | — | — |
| Water to 100 | BAL | BAL | BAL | BAL | BAL | BAL | BAL | BAL | BAL |
| pH | 2.6 | 2.6 | 2.6 | 2.6 | 2.6 | 2.87 | 2.7 | 2.6 | 2.6 |
| Straightening (%) | 67% | 93% | 91% | 97% | 87% | 93% | 93% | 95% | 91% |

When citraconic acid is added to the formulations, the straightening efficiency values increase substantially to values higher than 80% as seen in above Table III. Furthermore, Table 0.11 also shows that when citraconic acid is alone at 20%, and in combination with sodium diacetate as buffer, it yields a straightening efficiency lower than 70%. Both observations clearly indicate that the combination of citraconic acid with the selected aldehydes, ketones, and alcohols shown in the examples leads to hair straightening synergistic effects.

TABLE IV
Citraconic acid in combination with magnesium oxide
and hydrogen peroxide

| | EXP | EXP | EXP | EXP | EXP | EXP |
|---|---|---|---|---|---|---|
| Ingredients & | 22 | 23 | 24 | 25 | 26 | 27 |
| Evaluation | (wt. %) | (wt. %) | (wt. %) | (wt. %) | (wt. %) | (wt. %) |
| Citraconic acid | 20% | 20% | 20% | 20% | 20% | 20% |
| Butyraldehyde | 2.5% | — | — | — | — | — |
| Iso- | — | 2.5% | — | — | — | — |
| butyraldehyde | | | | | | |
| Propionaldehyde | — | — | 2.5% | — | — | — |
| Phenoxyethanol | — | — | — | 2.5% | — | — |
| Cyclopentanome | — | — | — | — | 2.5% | — |
| Glucose | — | — | — | — | — | 2.5% |
| Sodium | 10% | 10% | 10% | 10% | 10% | 10% |
| Diacetate | | | | | | |
| Isopropyl | — | — | — | — | — | — |
| alcohol | | | | | | |
| Propylene | — | — | — | — | — | — |
| glycol | | | | | | |
| Hydrogen | 0.6% | 0.6% | 0.6% | 0.6% | 0.6% | 0.6% |
| Peroxide | | | | | | |
| Magnesium | — | — | — | 0.25% | — | — |
| Oxide | | | | | | |
| Water to 100 | BAL | BAL | BAL | BAL | BAL | BAL |
| pH | 2.6 | 2.6 | 2.6 | 2.6 | 2.7 | 2.6 |
| % Straightening | 100% | 100% | 100% | 93% | 91% | 83% |

Table IV shows addition of magnesium oxide and hydrogen peroxide to combination of citraconic acid, butyraldehyde, and sodium diacetate enhanced hair straightening to 100%. The results clearly indicate that selective addition of actives in specific amounts lead to enhanced efficacy in hair straightening.

### Examples 28-31

TABLE V
Addition of oxide/peroxide to citraconic acid alone or its combinations

| Ingredients & | EXP 28 | EXP 29 | EXP 30 | EXP 31 |
|---|---|---|---|---|
| Evaluation | (wt. %) | (wt. %) | (wt. %) | (wt. %) |
| Citraconic acid | 20% | 20% | 20% | 20% |
| Butyraldehyde | 1.0% | — | — | — |
| Iso-butyraldehyde | — | 1.0% | — | — |
| Propionaldehyde | — | — | 1.0% | — |
| Phenoxyethanol | — | — | — | — |
| Cyclopentanone | — | — | — | 1.0% |
| Glucose | — | — | — | — |
| Sodium Diacetate | 10% | 10% | 10% | 10% |
| Isopropyl alcohol | — | — | — | — |
| Propylene glycol | — | — | — | — |
| Hydrogen peroxide | 0.6% | 0.6% | 0.6% | 0.6% |
| Magnesium oxide | 0.25% | 0.25% | 0.25% | 0.25% |
| Water to 100 | BAL | BAL | BAL | BAL |
| pH | 2.6 | 2.6 | 2.6 | 2.7 |
| % Straightening | 100% | 100% | 100% | 84% |

Table V shows, on the other hand, that the addition of magnesium oxide and hydrogen peroxide to a combination of citraconic acid, butyraldehyde, and sodium diacetate enhanced hair straightening to 100%. The results clearly indicate that selective addition of actives in specific amounts lead to enhanced efficacy in hair straightening.

### Example 32: Formulation of Cream A: Cream and Activator are Formulated Separately

| | |
|---|---|
| Ingredient | Quantity (wt. %) |

Part 1: Cream Formula A

| | |
|---|---|
| Sodium diacetate | 10.1 |
| Ethylene carbonate | 3.8 |
| Stearyl alcohol | 3.5 |
| Cyclo pentasiloxane | 2.9 |
| Cetyl alcohol | 2.9 |
| Dimethicone | 2.3 |
| Aspartic acid | 1.8 |
| Quaternium-18 | 1.5 |
| Thioglycolic acid | 1.1 |
| DMDM Hydantoin | 0.6 |
| Fragrance | 0.5 |
| Propylene Glycol | 0.3 |
| Butylene Glycol | 0.2 |
| Water | 68.5 |

Part II: Activator A-1

| | |
|---|---|
| Citraconic acid | 60.0 |
| Water | 40.0 |

Mode of preparation: Cream A (75%) was taken and added to activator A-1 (25%) followed by thorough mixing. The mixture was applied to hair and left for 30 minutes. Hair was cleaned with towel, blow dried, and treated with hot iron.

### Example 33: Formulation of Cream Ab and Anhydrous Paste Activator Ab-1

| | |
|---|---|
| Ingredient | Quantity (wt. %) |

Part 1: Cream Formula Ab

| | |
|---|---|
| Sodium diacetate | 10.1 |
| Stearyl alcohol | 3.5 |
| Cetyl alcohol | 2.9 |
| Aspartic acid | 1.8 |
| Quaternium-18 | 1.5 |
| DMDM Hydantoin | 0.6 |
| Fragrance | 0.5 |
| Propylene Glycol | 0.3 |
| Butylene Glycol | 0.2 |
| Water | 78.6 |

Part II: Anhydrous Paste Activator Ab-1

| | |
|---|---|
| Citraconic acid | 89.0 |
| Urea Peroxide | 10.0 |
| Dimthicone | 1.0 |

Mode of preparation: 30.0% ofAnhydrous Activator Ab-1 is added to 70% ofCream Ab followed by thorough mixing. The mixture was applied to hair and left for 30 minutes. Hair was cleaned with towel, blow dried, and treated with hot iron.

### Example 34: Formulation of Mousse: Mousse and Activator were Prepared Separately

Part 1: Mousse base Formula B

| | Quantity (wt. %) | |
|---|---|---|
| Ingredient | A | B |
| Sodium diacetate | — | 10.1 |
| Ethylene carbonate | — | 4.01 |
| Thioglycolic acid | — | 1.01 |
| PEG-7 Glycerol Cocoate | 8.01 | 8.01 |
| Xiameter OFX 0193 | 2.51 | 2.51 |
| Prolipid 161 | 3.78 | 3.78 |
| Natrosol Plus 330 CS | 1.51 | 1.51 |
| Benzaldehyde | — | 0.02 |
| 5% NaOH | 1.51 | — |
| Water | 82.68 | 69.14 |

Part II: Activator B-1

| Ingredient | Quantity (%) |
|---|---|
| Citraconic acid | 60.0 |
| Water | 40.0 |

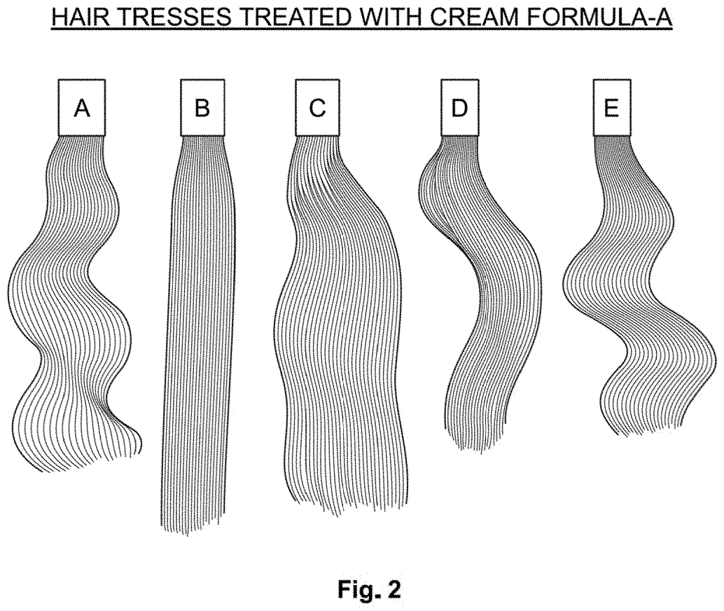

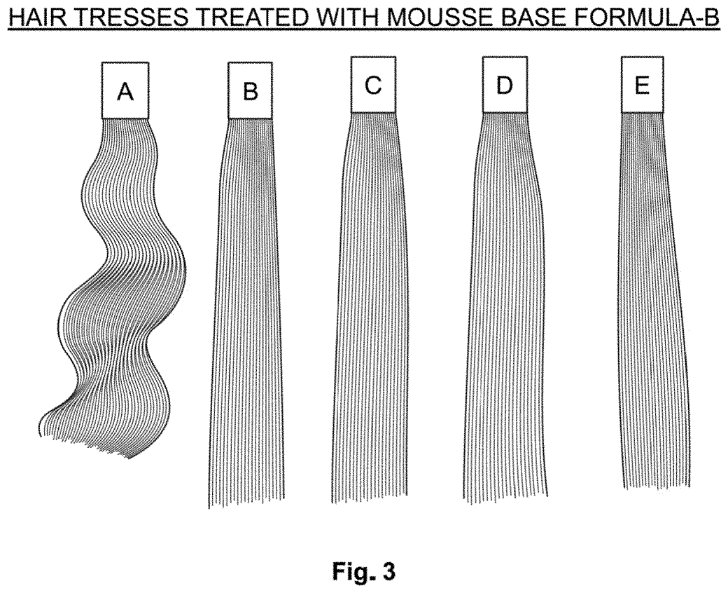

Mode of preparation—mousse base (75 wt. %) as described above was taken and added to activator B-1 (25 wt. %) followed by thorough mixing of the contents. Mousse so formed was applied onto the hair from pump and left for 30 min. Later, hair was cleaned with towel, blow dried, and treated with hot iron. For comparative studies, FIGS. 2 and 3 are shown hair swatches straightened with citraconic acid under different conditions as indicated above in Example 27.

FIG. 2 shows the effects of shampoo washes on hair straightened using just citraconic acid and neutralized to a pH of 2.5 with a dilute solution of NaOH and with no additional ingredients (Formulation A+Activator B-1). FIG. 3 shows, on the other hand, effects of hair tresses straightened at the same pH with a select blend of ingredients (Formulation B+Activator B-1), current inventive element of the present invention. An inspection of these two Figures reveals that when citraconic acid (Activator B-1) is combined with mousse formulation A, the level of hair straightening decreases as the number of shampoo washings increases. In contrast, when citraconic acid (Activator B-1) is combined with mousse formulation B containing the reducing agent thioglycolic acid, the solvent ethylene carbonate, and the buffer sodium diacetate, the level of performance is maintained for 20 shampoo washings.

Although the present application is shown and described with respect to certain aspects, it is obvious that various modifications will become apparent to those skilled in the art upon reading and understanding the specification and the appended claims. The present application includes all such improvements and modifications and is limited only by the scope of the claims.

What is claimed is:

1. A hair straightening or hair styling composition comprising:
(i) from 0.5 to 60 wt. % of citraconic acid;
(ii) (a) from 0.01 to 5 wt. % of a reducing agent; and/or (b) from 0.001 to 50 wt. % of (b1) an aldehyde, (b2) a thioacetal, (b3) a ketone, (b4) an alcohol or combinations thereof;
(iii) from 0.1 to 20 wt. % of a buffering agent;
(iv) from 0.01 to 5 wt. % of an oxidizing agent:
(v) from 0.0 to 5 wt. % of a carboxylic or a dicarboxylic acid; and
(vi) from 2 to 20 wt. % of at least one solvent.

2. The hair straightening or hair styling composition according to claim 1, wherein the composition has a pH of 1.5 to 3.0.

3. The hair straightening or hair styling composition according to claim 1, wherein the composition has a pH of 2.0 to 2.7.

4. The hair straightening or hair styling composition according to claim 1, wherein the composition has a pH of 2.0 to 2.57.

5. The hair straightening or hair styling composition according to claim 1, wherein the composition shows at least 60% efficacy of straightening or styling lasting for 20 shampoo wash cycles.

6. The hair straightening or hair styling composition according to claim 1, wherein the reducing agent is selected from the group consisting of thioglycolic acid and thiolactic acid and salts thereof, sodium bisulfite, sodium sulfite, cysteine and thioacetals.

7. The hair straightening or hair styling composition according to claim 1, wherein the aldehyde is selected from the group consisting of acetaldehyde, propionaldehyde, butyraldehyde, iso-butyraldehyde, glyoxal, glutaraldehyde, pyruvaldehyde, methylglyoxal di-acetate, glyoxylic acid, 5-methyl furaldehyde, cinnamaldehyde, benzaldehyde, hydroxyaldehyde, cyclohexane carbaldehyde, (R)-2-chloro-3-phenylpropanal, 2-methyl-3-oxopentanal, 4-methyl-3-oxopentanal, 2-(3-ethylbenzoyl)-3-oxopentanal, 2,2,4-trimethyl-3-oxopentanal, 2,2-dimethyl-3-oxopentanal, 4-oxopentanal, anisaldehyde, hemiacetals, and thio acetals.

8. The hair straightening or hair styling composition according to claim 1, wherein the ketone is selected from the group consisting of acetone, mono-hydroxyacetone, dihydroxy acetone, cyclopentanone, hexanone, caprolactone, valerolactone, methyl furyl ketone, methyl isobutyl ketone and raspberry ketone.

9. The hair straightening or hair styling composition according to claim 1, wherein the alcohol is selected from the group consisting of phenoxyethanol, ethanol, methanol, propanediol, propylene glycol, octane diol and butylene glycol.

10. The hair straightening or hair styling composition according to claim 1, wherein the buffering agent is selected from the group consisting of sodium diacetate, sodium acetate, sodium succinate, disodium succinate, sodium citrate, sodium formate, and sodium or ammonium salts.

11. The hair straightening or hair styling composition according to claim 1, wherein the oxidizing agent is selected from the group consisting of hydrogen peroxide, benzoyl peroxide, magnesium peroxide, calcium peroxide, dicumyl peroxide, urea peroxide, sodium persulfate, ammonium persulfate, sodium percarbonate, sodium bromates, cyclopentanone, magnesium oxide and zinc oxide.

12. The hair straightening or hair styling composition according to claim 1, wherein the solvent is selected from group consisting of isopropyl alcohol, propylene glycol, propanediol, glycerin, methyl acetate, ethanol, acetone, amyl acetate, ethyl dimethyl dioxolane, ethylene carbonate, acetyl-methylfurfural, butylene glycol, PEG200, PEG400, diethylene glycol and trimethyl phosphate.

13. The hair straightening or hair styling composition according to claim 1, wherein the composition is formulated into a cream, a mousse, a foam, a serum, a lotion paste, a hair gel, a hair pudding, or a spray.

14. The hair straightening or hair styling composition according to claim 13, wherein the formulation comprises at least one cosmetically acceptable ingredient selected from a cationic, anionic, or non-ionic polymer, a conditioning agent an oil, an emollient, a humectant, a petrolatum thickener, a mineral oil, an emulsifier, a fragrance, a keratin, a protein or a preservative.

15. The hair straightening or hair styling composition according to claim 1, wherein the composition further comprises an ionic, non-ionic, or cationic surfactant.

16. A hair straightening or hair styling composition comprising:
(i) from 0.5 to 60 wt. % of citraconic acid;
(ii) (a) from 0.01 to 8 wt. % of a reducing agent selected from thioglycolic acid, and thiolactic acid and salts thereof, or bisulfites; and/or (b) from 0.001 to 50 wt. % of (b1) an aldehyde selected from butyraldehyde, iso-butyraldehyde, benraldehyde, anisaldehyde, and propionaldehyde; (b2) thioacetal; (b3) a ketone selected from cyclo-pentanone or (b4) an alcohol selected from isopropyl alcohol and propylene glycol;
(iii) from 0.1 to 20 wt. % of a buffering agent selected from sodium diacetate, sodium acetate, and disodium succinate;
(iv) from 0.01 to 5 wt. % of an oxidizing agent selected from hydrogen peroxide, urea/peroxide, calcium peroxide and magnesium oxide;
(v) from 0.0 to 5 wt. % of a carboxylic or dicarboxylic acid; and
(vi) from 2 to 20 wt. % of at least one solvent selected from ethylene carbonate and water.

17. A method of straightening or styling hair, the method comprising:
(a) washing hair tresses with shampoo;
(b) drying the hair with a blow dryer in a temperature range from 40-80° C.;
(c) applying to hair the hair styling or hair straightening composition comprising: (i) from 0.5 to 60 wt. % of citraconic acid; (ii)(a) from 0.01 to 5 wt. % of a reducing agent; and/or (b) from 0.001 to 50 wt. % of (b1) an aldehyde, (b2) a thioacetal, (b3) a ketone, (b4) an alcohol, or combinations thereof; (iii) from 0.1 to 20 wt. % of a buffering agent; (iv) from 0.01 to 5 wt. % of an oxidizing agent; (v) from 0.0 to 5 wt. % of a carboxylic or a dicarboxylic acid; (vi) from 2 to 20 wt. % of a solvent;
(d) leaving the applied hair composition onto the hair for a period of 20 to 40 min;
(e) removing the excess formulation from the hair;
(f) applying from 5 to 15 hot ironing strokes to the hair, wherein the hot iron is set above the hair glass transition temperature (145° C.) and denaturation temperature (156° C.), between 160 to 220° C.;
(g) optionally, washing the hair with a shampoo; and
(h) obtaining the straightened or styled hair.

18. The method according to claim 17, wherein said hair styling or hair straightening composition is formulated in separate parts I and I; part 1 is activator comprising citraconic acid, part 11 is base formula and both parts are combined before use.

19. The method according to claim 17, wherein said hair styling or hair straightening composition is in the form of a cream, a foam, a serum, a lotion paste, a hair gel, a hair pudding, a spray, or a mousse.

20. The method according to claim 19, wherein the hair tresses are Caucasian, Asian, or African in origin selected from fine, medium and coarse virgin, damaged, colored, grey or bleached hair.

21. The method according to claim 20, wherein the hair tresses to be styled or straightened have visibly wavy, curly, tightly curly or kinky configuration.
